(19) United States
(12) Patent Application Publication
(10) Pub. No.: US 2007/0122894 A1
(43) Pub. Date: May 31, 2007

(54) Sterile microbiological nutrient media device and methods of using

(76) Inventor: Linda J. Richardson Casella, Neuilly sur Seine (FR)

Correspondence Address: Brian D. Voyce, 8401 Sterling Bridge Road, Chapel Hill, NC, 27516, US

(21) Appl. No.: 11/287,791

(22) Filed: Nov. 28, 2005

(51) Int. Cl. C12M 1/00; C12N 1/20; C12N 5/00

(52) U.S. Cl. 435/253.6; 435/404; 435/283.1

(57) ABSTRACT

The present invention relates to a device for assisting in the preparation of a sterile, microbiological nutrient media of a predetermined concentration. In general, such a device comprises three elements. An outer flexible envelope has a re-sealable opening. This outer envelope is comprised of a material that does not transmit light or water gases. At least one flexible media envelopes is disposed within the outer envelope, each media envelope having a re-sealable opening and a sterile interior, Each media envelope is comprised of a material that can be subjected to mechanical mixing forces. A predetermined amount of a sterile, particulate, microbiological nutrient media is disposed within each flexible media envelope. The media particles have a shape and configuration that allows the particles to settle within the media envelope. The predetermined amount of media is such that the media envelope is not overfilled when filled with liquid sufficient to constitute the predetermined concentration.

### BACKGROUND OF THE INVENTION

(1) Field of the Invention

The present invention relates to a device for assisting in the preparation of a sterile, microbiological nutrient media of a predetermined concentration or dosage. In general, such a device comprises three elements. An outer flexible envelope has a re-sealable opening. This outer envelope is comprised of a material that does not transmit light or fluids, such as water or oxygen. At least one flexible media envelope is disposed within the outer envelope, each media envelope having a re-sealable opening and a sterile interior. Each media envelope is comprised of a material that can be subjected to mechanical mixing forces. A predetermined amount of a sterile, particulate, microbiological nutrient culture media is disposed within each flexible media envelope. The media particles have a shape and configuration that allows the particles to settle within the media envelope. The predetermined amount of media is such that the media envelope does not overflow when filled with liquid sufficient to reconstitute the predetermined concentration.

An advantage of the present invention is that by providing the convenience and reliability of the media preparation with a one-step process, one can reduce the amount of labor involved in preparing medias for use, and therefore reduce the incidence of error. With the present invention, microbiology laboratories can be assured of a sterile media that can be readily prepared just in time for use.

(2) Description of the Related Art, Including Information Disclosed Under 37 CFR 1.97 & 1.98

Common practice today is for microbiology laboratories to prepare microbiological nutrient media in a batch method using conventional sterile containers as needed for a particulate test Typically, media is prepared by measuring media weight according to manufacturers recommendations, placing the media in a glass container; adding sterile water, placing the container on a heating unit; heating and agitating the media until the boiling point is reach, and the media is completed dissolved. Then that prepared media is placed in a disposable sterile bag, such as one disclosed in U.S. Pat. No. 5,564,829, along with a sample to be tested. The sample and media can be mixed in a mechanical mixing device and incubated prior to testing.

### BRIEF SUMMARY OF THE INVENTION

Methods and devices to provide sterile microbiological media of predetermined concentrations are important not only for providing convenience, but also for reducing the labor costs and test error. This is particularly advantageous for small and medium size laboratories with limited technical resources and where the irregular demand for media preparation usually results in inefficient use of laboratory personnel time and error in preparation of the media.

An object of the invention is to provide a high level of reproducibility and reliability in the preparation of sterile microbiological culture media a means for reliably making sterile microbiological media as needed in an error-free manner.

A second object of the invention is to be able to reduce the amount of storage space for media that is being held in inventory in a laboratory. By reconstituting media in predetermined unit amounts that correspond to particular tests, one can save inventory space and reduce costs. Media remains fresh until use, not being exposed to light and air each time the media container is open.

A third object of the present invention is to provide an ease of handling media throughout the laboratory. One device can be used for preparation, sample introduction and preparation, incubating

A fourth object of the invention is to provide a device not only for making a sterile microbiological media, but also for disposing of the media conveniently and safely after use. The present invention provides a one-step process for preparation of the media and a one-step disposal procedure which does not involve the risk of removing the mixture of media and contaminating microorganism from the device. Thus, the risk of cross contamination in the laboratory is reduced and the laboratory labor times are reduced. An additional benefit can be that the means for hazardous waste disposal is provided by the device as well.

The present invention is a device for assisting in the preparation of a sterile, microbiological media of a predetermined concentration. An outer flexible envelope has a re-sealable opening. The outer envelope is comprised of a material that does not transmit light or water (and preferably oxygen). At least one flexible media envelope is disposed within the outer envelope. Each media envelope has a re-sealable opening and a sterile interior. Each media envelope is comprised of a material that can be subjected to and withstand conventional mechanical mixing forces.

A predetermined amount of a sterile, particulate, microbiological media (such as a nutrient culture media) is disposed within each flexible media envelope. The media particles have a shape and configuration that allows the particles to settle within the media envelope. The predetermined amount of media is also configured such that the media envelope does not overflow when filled with liquid sufficient to reconstitute the predetermined concentration.

The present invention also comprises the methods for using the above devices. Fro example, one can prepare a microbiological nutrient culture media of a predetermined concentration using the following steps. First, one selects a device as described above. Next, one opens the outer flexible envelope and removes one flexible media envelope. Access to the sterile interior of the envelope is gained by opening the re-sealable opening of the removed flexible media envelope. Finally one deposits sterile, pre-filtered water in the opened flexible media envelope in a quantity sufficient to constitute the predetermined concentration.

The present devices can further be used to prepare an incubated microbiological sample for microbiological testing. One prepares the microbiological nutrient culture media as described above. Having done so, one deposits a microbiological sample into the opened flexible media envelope. Next one re-seals the opened flexible media envelope. The envelope containing media and sample is ready for thorough mixing using mechanical mixing devices, if desired. Finally, one incubates the re-sealed flexible media envelope under conditions suitable for growth of a microbe that is supported by the media.

### BRIEF DESCRIPTION OF THE DRAWINGS

FIG. 1 is an overhead view of the present invention showing the combination of the outer envelope and the inner flexible media envelope.

FIG. 2 is a perspective view of FIG. 1 prior to the full insertion and sealing of the flexible media envelope in the outer envelope.

FIG. 3 is a sectional view of the flexible media envelope.

FIG. 4 is an overhead view of the flexible media envelope.

FIG. 5 is an overhead view of the flexible media portion of the present invention showing a compartmented preferred embodiment.

FIG. 6 is a sectional view of FIG. 5.

FIG. 7 is an overhead view of the flexible media portion of the present invention showing a preferred embodiment having an inserted cachet.

### DETAILED DESCRIPTION OF THE INVENTION

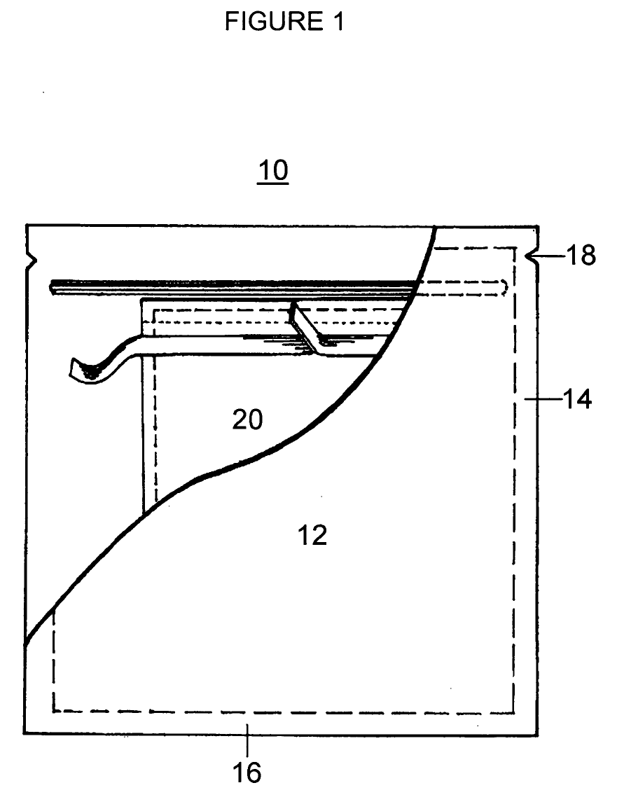

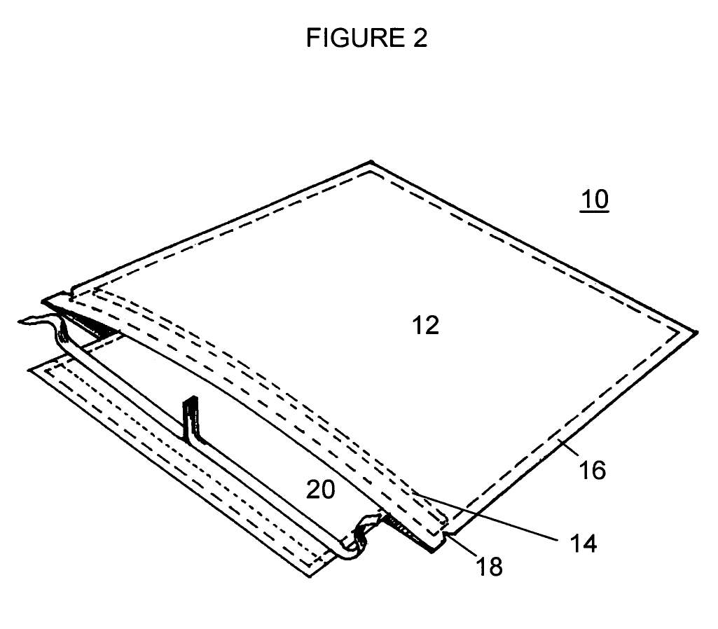

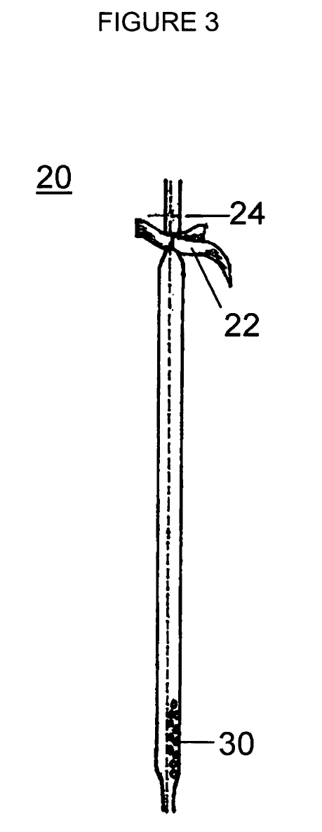

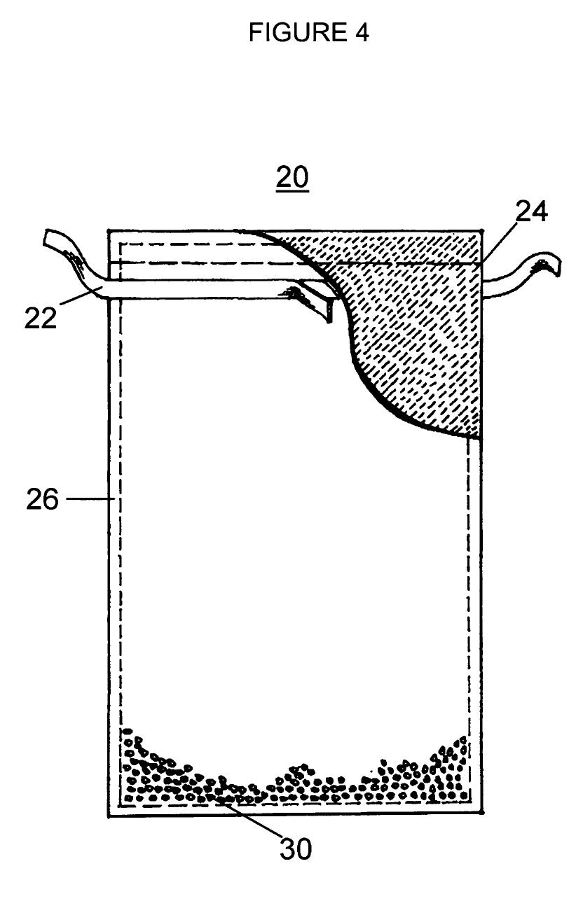

In preferred embodiments shown in FIGS. 1 to 4, the present invention relates to a device (10) for assisting in the preparation of a ready-to-use, sterile, microbiological nutrient culture media of a predetermined concentration. An outer flexible envelope (12) has a re-sealable opening (14). This outer envelope is comprised of a material that does not transmit light or water (and preferably oxygen). Suitable materials known to those of ordinary skill in the art include aluminized polymer films, such as polyester, polypropylene, or polyethylene films. Typically, the outer envelope will be made from two plies of film heat sealed in a rectilinear fashion (16) around the edges. A conventional tamper-proof opening (such as a tear line (18)) can be attached to one edge of the envelope.

The tamperproof opening is disposed such that the tamper proof opening must be opened before the interior of the outer envelope can be accessed. The tamper proof opening serves to indicate that the device as received has remained unopened since manufacture. In addition, one can provide a conventional re-sealable grooved channel opening also to one edge of the envelope or bag. If combined with the tamper proof opening, then the tamper proof opening should be proximal to the edge and the resealable opening distal to that same edge.

At least one flexible media envelope (20) is disposed within the outer envelope. Each culture media envelope has a re-sealable opening (22) and a sterile interior. Each media envelope is comprised of a material that can be subjected to and withstand conventional mechanical mixing forces, such as those applied by a stomacher or similar types of mechanical mixing equipment. Suitable materials known to those of ordinary skill in the art include transparent or translucent polymer films, such as polyester, polypropylene, polyethylene films, preferably with a thickness range of 2 to 4 mils. Typically, the flexible media envelope will be made from two plies of film heat sealed in a rectilinear fashion around the edges (26), or as a tube sleeve sealed at top and bottom edges, as disclosed in U.S. Pat. No. 5,564,829 to Lafond.

Preferably, the flexible media envelope will have a tamper proof opening (24) which can be attached to one edge of the envelope or bag. The re-sealable opening, either a twirl-type re-sealable opening or a conventional grooved channel and an insertable channel member opening (often referred to as a zip-loc closure) is disposed below the tamper-proof opening. The tamper-proof opening on the flexible media envelope serves to indicate that the particular media envelope as received has remained unopened since manufacture, even if the outer envelope has been opened. The envelope material should be selected such that the static charges and other surfaces of the material allow for particles to settle into the envelope, as opposed to clinging, flying, or dusting away from the surface. Also, the flexible media envelope can be dimensioned and configured such that the flexible media envelope is self-supporting, i.e., can stand upright on a laboratory bench surface prior to or after filling.

Preferably, the flexible media envelope material is selected so that it can be sterilized with further processing, such as being irradiated with sterilizing radiation from a conventional gamma radiation device, and still maintain the described properties. Not only can the flexible media envelope be irradiated after fabrication in the device for sterility assurance, but the user can re-seal the used device and sterilize the device with sample (such as by autoclaving) prior to disposal.

A predetermined amount of a sterile, particulate, microbiological nutrient media is disposed within each flexible media envelope. The media particles have a shape and configuration that allows the particles to settle within the media envelope. For example, one can use granulated or agglomerated media as opposed to fine powders that have a high static force, such as a standard fine powdered dehydrated culture media. Examples of dehydrated media formulations classical nutrient media that can be used in the present invention include buffered peptone water, lactose broth, or Listeria enrichment medium.

The predetermined amount of media used in the present invention is configured such that the media envelope does not overflow when filled with liquid sufficient to reconstitute the predetermined concentration.

Preferably, one will provide a fill indicator means, such as a line that is marked on the outside of the flexible media envelope that traverses across the width of the bag. The user simply fills the bag with the sterile liquid up to the indicated fill point. The fill line should allow for the compression of the flexible media in a mechanical mixer such as a stomacher. One can also provide for information being placed on the bag either through direct printing on the envelope or an applied adhesive label.

Kits can be made that incorporate the above nutrient media device along with an accompanying package of sterile liquid that comprises a volume that is sufficient to reconstitute the media in the device to the predetermined media concentration. The liquid and the also media supplements can be in a conventional sterile packaging forms.

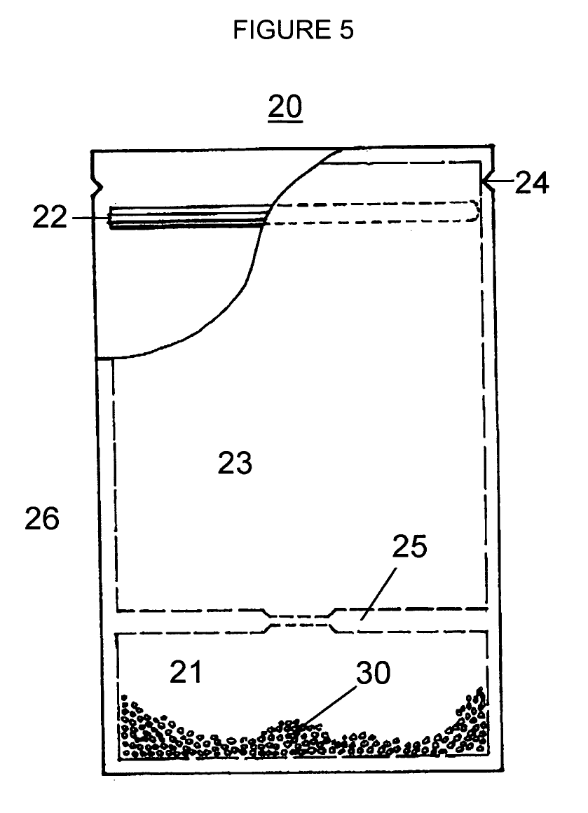

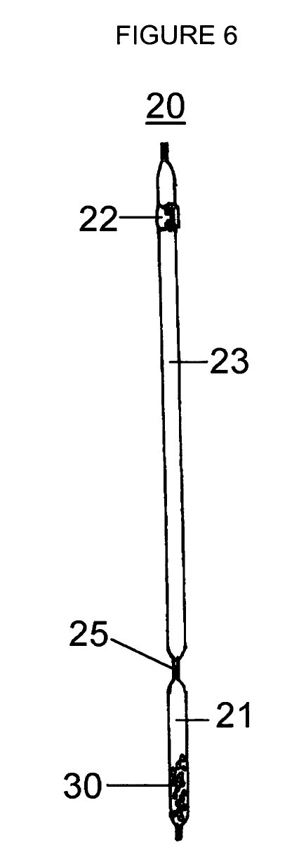

In another preferred embodiment shown in FIGS. 5 and 6, the flexible media envelope can have more than one compartments or partitions. For example, one can have a first compartment (21) and a second compartment (23). The first compartment has a predetermined amount of media disposed therein. A partition (25) between the two compartments is designed and configured to allow communication between the two compartments without having to open the flexible media envelope. For example, a heat sealed seam can have a weaker portion that is designed to open either by applying a tensile force to the seam or by manipulating the contents within one of the compartments. The second compartment can contain either a solid or a liquid that is media-related to either the incubation of a microbial sample or the testing of such a sample. It may also contain the predetermined amount of liquid necessary to reconstitute the media. One reconstitutes the media by opening the partition seam or divider allowing the mixing of media and liquid.

Alternatively, the flexible media envelope can contain a sterile releasing matrix disposed within the interior of the flexible media envelope. The releasing matrix, such as a dissolvable film or a porous membrane has a media-related component disposed therein. Upon contacting the releasing matrix with the reconstituting liquid, the media-related component is freed from the releasing matrix.

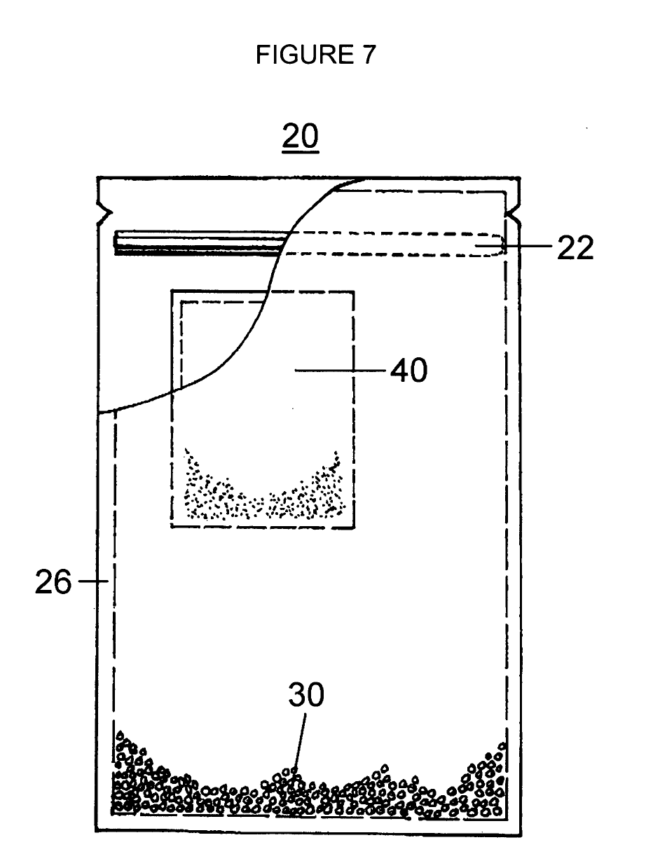

In yet another preferred embodiment shown in FIG. 7, the flexible media envelope (20) can have a pouch or cachet (40) inserted into the interior if the flexible media envelope. The cachet can contain a sterile media-related liquid or solid, or the above-descirbed releasing matrix. By manipulating the cachets within the flexible media envelope, one can mix the media and either media supplements or reconstituting liquids without removing the sterile cachets or pouches from the flexible media envelope. For example, one can have an envelope that is filled with the predetermined amount of media (30). A cachet containing a media supplement is placed within the flexible media envelope. The dehydrated media has no contact with the supplement. A second pouch filled with the predetermined amount of liquid for reconstituting the media is also placed within the flexible media envelope. Again the liquid has no contact with the media. The interior of the entire device, including the pouches and cachet interiors, is sterile

The ordinarily skilled artisan can appreciate that the present invention can incorporate any number of the preferred features described above.

All publications or unpublished patent applications mentioned herein are hereby incorporated by reference thereto.

Other embodiments of the present invention are not presented which are obvious to those of ordinary skill in the art, now or during the term of any patent issuing from this patent specification, and thus, are within the spirit and scope of the present invention.

We claim:

1. A device for assisting in the preparation of a sterile, microbiological media of a predetermined concentration comprising:
a) an outer envelope having a re-sealable opening, the outer envelope comprised of a material that does not transmit oxygen, light or water;
b) at least one flexible media envelope disposed within the outer envelope, each media envelope having a re-sealable opening and a sterile interior and each media envelope being comprised of a material that can be subjected to mechanical mixing forces; and
c) a predetermined amount of a sterile, particulate, microbiological media disposed within each flexible media envelope, the media particles having a shape and configuration that allows the particles to settle within the media envelope and the predetermined amount of media being such that the media envelope is not overfilled when filled with liquid sufficient to reconstitute the predetermined concentration.

2. The media device of claim 1 wherein the outer envelope also comprises a tamper proof opening disposed such that the tamper proof opening must be opened before the interior of the outer envelope can be accessed.

3. The media device of claim 1 wherein each flexible media envelope also comprises a fill indicator means for the amount of sterile liquid introduced into the flexible media envelope.

4. The media device of claim 1 wherein a filter membrane is inserted into the flexible media envelope such that the interior of the media envelope is partitioned between a filtrate portion and a retentate portion.

5. The media device of claim 1 wherein the flexible media envelope has at least two compartments separated by an opening means, one of which is filled with the predetermined amount of media and the other filled with a liquid or solid that can be mixed with the predetermined amount of media.

6. The media device of claim 1 wherein the flexible media envelope has at least one pouch disposed in the interior of the sealed flexible media envelope, the pouch being filled with the predetermined amount of media and the interior of the flexible media envelope being filled with a liquid or solid that can be mixed with the predetermined amount of media.

7. The media device of claim 1 wherein the flexible media envelope has at least one pouch disposed in the interior of the sealed flexible media envelope, the pouch being filled with a liquid or solid that can be mixed with the predetermined amount of media and the interior of the flexible media envelope being filled with the predetermined amount of media.

8. A sterile microbiological culture media kit comprising:
a) a device for assisting in the preparation of a sterile, microbiological nutrient culture media of a predetermined concentration comprising:
i) an outer envelope having a re-sealable opening, the outer envelope comprised of a material that does not transmit oxygen, light or water;
ii) at least one flexible media envelope disposed within the outer envelope, each media envelope having a re-sealable opening and a sterile interior and each media envelope being comprised of a material that can be subjected to mechanical mixing forces;
iii) a predetermined amount of a sterile, particulate, microbiological nutrient media disposed within each flexible media envelope, the media particles having a shape and configuration that allows the particles to settle within the media envelope and the predetermined amount of media being such that the media envelope does not overflow when filled with liquid sufficient to reconstitute the predetermined concentration; and
b) a package of sterile liquid in a volume that is sufficient to reconstitute the media in the device to the predetermined media concentration.

9. The media kit of claim 8 wherein the outer envelope also comprises a tamper proof opening disposed such that the tamper proof opening must be opened before the interior of the outer envelope can be accessed.

10. The media kit of claim 8 wherein each flexible media envelope also comprises a fill indicator means for the amount of sterile liquid introduced into the flexible media envelope.

11. The media kit of claim 8 wherein a filter membrane is inserted into the flexible media envelope such that the interior of the media envelope is partitioned between a filtrate portion and a retentate portion.

12. The media kit of claim 8 wherein the flexible media envelope has at least two compartments separated by an opening means, one of which is filled with the predetermined amount of media and the other filled with a liquid or solid that can be mixed with the predetermined amount of media.

13. The media kit of claim 8 wherein the flexible media envelope has at least one pouch disposed in the interior of the sealed flexible media envelope, the pouch being filled with the predetermined amount of media and the interior of the flexible media envelope being filled with a liquid or solid that can be mixed with the predetermined amount of media.

14. The media kit of claim 8 wherein the flexible media envelope has at least one pouch disposed in the interior of the sealed flexible media envelope, the pouch being filled with a liquid or solid that can be mixed with the predetermined amount of media and the interior of the flexible media envelope being filled with the predetermined amount of media.

15. A method for preparing a microbiological culture media of a predetermined concentration comprising:
a) selecting a device comprised of:
i) an outer envelope having a re-sealable opening, the outer envelope comprised of a material that does not transmit oxygen, light or water;
ii) at least one flexible media envelopes disposed within the outer envelope, each media envelope having a re-sealable opening and a sterile interior and each media envelope being comprised of a material that can be subjected to mechanical mixing forces; and
iii) a predetermined amount of a sterile, particulate, microbiological media disposed within each flexible media envelope, the media particles having a shape and configuration that allows the particles to settle within the media envelope and the predetermined amount of media being such that the media envelope does not overflow is not overfilled when filled with liquid sufficient to constitute the predetermined concentration;
b) opening the outer flexible envelope and removing one flexible media envelope;
c) opening the re-sealable opening of the removed flexible media envelope; and
d) depositing sterile liquid in the opened flexible media envelope in a quantity sufficient to reconstitute the predetermined concentration.

16. The method for preparing a microbiological media of claim 15 wherein the outer envelope also comprises a tamper proof opening disposed such that the tamper proof opening must be opened before the interior of the outer envelope can be accessed.

17. The method for preparing a microbiological media of claim 15 wherein each flexible media envelope also comprises a fill indicator means for the amount of sterile liquid introduced into the flexible media envelope.

18. The method for preparing a microbiological media of claim 15 wherein a filter membrane is inserted into the flexible media envelope such that the interior of the media envelope is partitioned between a filtrate portion and a retentate portion.

19. The method for preparing a microbiological media of claim 15 wherein the flexible media envelope has at least two compartments separated by an opening means, one of which is filled with the predetermined amount of media and the other filled with a liquid or solid that can be mixed with the predetermined amount of media, and the media is reconstituted by allowing communication between the compartment through the opening means.

20. The method for preparing a microbiological media of claim 15 wherein the flexible media envelope has at least one pouch disposed in the interior of the sealed flexible media envelope, the pouch being filled with the predetermined amount of media and the interior of the flexible media envelope being filled with a liquid or solid that can be mixed with the predetermined amount of media, and the media is reconstituted by allowing communication between the pouch and the flexible media envelope interior.

21. The method for preparing a microbiological media of claim 15 wherein the flexible media envelope has at least one pouch disposed in the interior of the sealed flexible media envelope, the pouch being filled with a liquid or solid that can be mixed with the predetermined amount of media and the interior of the flexible media envelope being filled with the predetermined amount of media, and the media is reconstituted by allowing communication between the pouch and the flexible media envelope interior.

22. A method for preparing an incubated microbiological sample for microbiological testing comprising:
a) selecting a device comprised of:
i) an outer envelope having a re-sealable opening, the outer envelope comprised of a material that does not transmit oxygen, light or water;
ii) at least one flexible media envelopes disposed within the outer envelope, each media envelope having a re-sealable opening and a sterile interior and each media envelope being comprised of a material that can be subjected to mechanical mixing forces; and
iii) a predetermined amount of a sterile, particulate, microbiological media that can support growth of a microbe disposed within each flexible media envelope, the media particles having a shape and configuration that allows the particles to settle within the media envelope and the predetermined amount of media being such that the media envelope does not overflow is not overfilled when filled with liquid sufficient to constitute the predetermined concentration;
b) opening the outer flexible envelope and removing one flexible media envelope;
c) opening the re-sealable opening of the removed flexible media envelope;
d) depositing sterile liquid into the opened flexible media envelope in a quantity sufficient to reconstitute the predetermined concentration;
e) depositing a microbiological sample into the opened flexible media envelope;
f) re-sealing the opened flexible media envelope; and
g) incubating the re-sealed flexible media envelope under conditions suitable for growth of a microbe that is supported by the media.

23. The method for preparing an incubated microbiological sample for microbiological testing of claim 22 wherein the outer envelope also comprises a tamper proof opening disposed such that the tamper proof opening must be opened before the interior of the outer envelope can be accessed.

24. The method for preparing an incubated microbiological sample for microbiological testing of claim 22 wherein each flexible media envelope also comprises a fill indicator means for the amount of sterile liquid introduced into the flexible media envelope.

25. The method for preparing an incubated microbiological sample for microbiological testing of claim 22 wherein a filter membrane is inserted into the flexible media envelope such that the interior of the media envelope is partitioned between a filtrate portion and a retentate portion.

26. The method for preparing an incubated microbiological sample for microbiological testing of claim 22 wherein the flexible media envelope has at least two compartments separated by an opening means, one of which is filled with the predetermined amount of media and the other filled with a liquid or solid that can be mixed with the predetermined amount of media, and the media is reconstituted by allowing communication between the compartment through the opening means.

27. The method for preparing an incubated microbiological sample for microbiological testing of claim 22 wherein the flexible media envelope has at least one pouch disposed in the interior of the sealed flexible media envelope, the pouch being filled with the predetermined amount of media and the interior of the flexible media envelope being filled with a liquid or solid that can be mixed with the predetermined amount of media, and the media is reconstituted by allowing communication between the pouch and the flexible media envelope interior.

28. The method for preparing an incubated microbiological sample for microbiological testing of claim 22 wherein the flexible media envelope has at least one pouch disposed in the interior of the sealed flexible media envelope, the pouch being filled with a liquid or solid that can be mixed with the predetermined amount of media and the interior of the flexible media envelope being filled with the predetermined amount of media, and the media is reconstituted by allowing communication between the pouch and the flexible media envelope interior.

29. The media device of claim 1 where in the flexible media envelope comprises at least a first compartment and a second compartments separated by a partition, the partition being able to allow communication between the two compartments without opening the flexible media envelope.

30. The media device of claim 29 wherein the reconstituting liquid is in one of the compartments and the particulate media is in the other compartment.

31. The media device of claim 1 wherein a cachet is disposed within the interior of the flexible media envelope, the cachet containing either a media-related liquid or a media-related solid.

32. The media device of claim 1 wherein a releasing matrix is disposed within the interior of the flexible media envelope, the releasing matrix having an associated media-related component and whereby the media-related component is released from the releasing matrix upon contact with the reconstituting liquid.
